(19) United States
(12) Patent Application Publication
(10) Pub. No.: US 2024/0423790 A1
(43) Pub. Date: Dec. 26, 2024

(54) ACTUATION MECHANISM FOR A PROSTHETIC HEART VALVE

(71) Applicant: EDWARDS LIFESCIENCES CORPORATION, Irvine, CA (US)

(72) Inventors: Anatoly Dvorsky, Haifa (IL); Michael Bukin, Pardes Hanna (IL); Nikolai Gurovich, Hadera (IL); Eran Grosu, Kfar-Saba (IL)

(21) Appl. No.: 18/825,316

(22) Filed: Sep. 5, 2024

Related U.S. Application Data
(63) Continuation of application No. PCT/US2023/014795, filed on Mar. 8, 2023.
(60) Provisional application No. 63/318,547, filed on Mar. 10, 2022.

(51) Int. Cl. A61F 2/24

(52) U.S. Cl. CPC A61F 2/2418; A61F 2/243

(57) ABSTRACT

This disclosure is directed to prosthetic heart valves and actuators for prosthetic heart valves. In some examples, the actuator can comprise an internally threaded bore that engages an externally threaded portion of the frame component, such as a stationary post. In some examples, the actuator can comprise an externally threaded end portion that engages an internally threaded bore in a frame component, such as a stationary post.

### CROSS REFERENCE TO RELATED APPLICATIONS

This application is a continuation of International Patent Application No. PCT/US2023/014795, filed on Mar. 8, 2023, which claims the benefit of U.S. Provisional Patent Application No. 63/318,547, filed on Mar. 10, 2022, both of which applications are incorporated by reference herein.

### FIELD

The present disclosure relates to implantable expandable prosthetic heart valves and actuation mechanisms for use with expandable prosthetic heart valves.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (for example, stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient's vasculature (for example, through a femoral artery and the aorta) until the prosthetic heart valve reaches the implantation site in the heart. The prosthetic heart valve is then expanded to its functional size, for example, by inflating a balloon on which the prosthetic valve is mounted, actuating a mechanical actuator that applies an expansion force to the prosthetic heart valve, or by deploying the prosthetic heart valve from a delivery capsule of the delivery apparatus so that the prosthetic heart valve can self-expand to its functional size.

These prosthetic heart valves are subject to various technical challenges. To facilitate the delivery of the valve to the desired implantation site, it is desirable for these prosthetic heart valves to have a small cross-sectional profile or crimp profile. Additionally, it is desirable for the components of the prosthetic heart valve to be robust and protected from wear and fatigue to support prolonged periods of use without damage or failure.

Mechanical actuators for radially expanding transcatheter heart valves can increase the overall crimp profile of the prosthetic and/or can increase the complexity and cost of the manufacturing process. Thus, there is a continuing need for new and improved transcatheter prosthetic heart valves having mechanical actuators.

### SUMMARY

Disclosed herein are prosthetic heart valve assemblies including actuation bolts engaging directly with one or more struts of the prosthetic heart valve frame. The disclosed actuation bolts can include an internally threaded bore and can operationally engage with an externally threaded end portion of a strut of the prosthetic heart valve. Alternatively, the disclosed actuation bolts can include an externally threaded end portion that can operationally engage with an internally threaded bore in a strut of the prosthetic heart valve. Such actuation assemblies can avoid the need for an actuation nut. This in turn avoids the need for a nut window in the struts of the prosthetic heart valve operationally engaged with the actuation mechanism. As such, the actuation assemblies disclosed herein enable the design of prosthetic heart valves with narrower crimp profiles and which avoid wear from the relative motion of an actuation nut against the struts of the prosthetic heart valve.

A prosthetic heart valve can comprise a frame and a valvular structure coupled to the frame. In addition to these components, a prosthetic heart valve can further comprise one or more of the components disclosed herein.

In some examples, a prosthetic heart valve can comprise a sealing member configured to reduce paravalvular leakage.

In some examples, a prosthetic heart valve can comprise an actuator comprising an internally threaded bore.

In some examples, the internally threaded bore can receive an externally threaded portion of a post.

In some examples, a prosthetic heart valve can comprise an actuator comprising an externally threaded portion.

In some examples, the externally threaded portion of the actuator can be received by an internally threaded bore in a post.

Certain examples concern a prosthetic valve, comprising a radially expandable frame with a plurality of interconnected struts, wherein the frame is radially expandable between a radially compressed state and a radially expanded state and the frame comprises at least one axially oriented first post having an externally threaded portion. The prosthetic heart valve also includes a valvular structure disposed within the frame and configured to regulate the flow of blood through the frame in one direction and at least one rotatable actuator operatively coupled to the frame. The actuator comprises an internally threaded bore that receives the externally threaded portion of the post, wherein the actuator is rotatable relative to the post in a first rotational direction to produce radial expansion of the frame from the radially compressed state to the radially expanded state.

Certain examples concern a prosthetic valve, comprising an annular frame with a first end portion, a second end portion, a longitudinal axis extending from the first end portion to the second end portion, and a plurality of struts forming a plurality of cells between the first end portion and the second end portion. The prosthetic valve also includes a valvular structure disposed within the frame and configured to regulate the flow of blood through the frame in one direction. The prosthetic valve also includes a plurality of vertical posts oriented parallel to the longitudinal axis and an actuation member with a first end portion and a second end portion, wherein the first end portion comprises an axially oriented, internally threaded bore. The internally threaded bore of the actuation member is operatively coupled to an externally threaded portion of a first vertical post of the plurality of vertical posts. The actuation member is rotatable relative to the first vertical post in a first rotational direction to radially expand the frame from a radially compressed state to a radially expanded state and rotatable in a second rotational direction to radially compress the frame from the radially expanded state to the radially compressed state.

Certain examples concern a medical assembly, comprising a prosthetic heart valve with a radially expandable frame, a valvular structure disposed within the frame and configured to regulate the flow of blood through the frame in one direction, a vertically oriented first post, and at least one actuator operatively coupled to the vertically oriented first post. The medical assembly also includes a delivery device releasably attached to the actuator. The frame comprises a plurality of interconnected struts and is radially expandable between a radially compressed state and a radially expanded state. The vertically oriented first post comprises an externally threaded end portion. The actuator comprises an internally threaded end portion that receives the externally threaded end portion of the vertically oriented first post, wherein the actuator is rotatable relative to the first post in a first rotational direction to produce radial expansion of the frame.

In some examples, a prosthetic heart valve comprises one or more of the components recited in Examples 1-36 below.

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1A is a perspective view of one example of a prosthetic valve including a frame and a plurality of leaflets attached to the frame.

FIG. 1B is a perspective view of the prosthetic valve of FIG. 1A with an outer skirt disposed around the frame.

FIG. 2A is a perspective view of a frame for the prosthetic valve of FIG. 1A.

FIG. 2B is a front portion of the frame shown in FIG. 2A.

FIG. 3 is a side elevation view of a delivery apparatus for a prosthetic device, such as a prosthetic valve, according to one example.

FIG. 4 is a perspective view of a portion of an actuator of the prosthetic device of FIGS. 1-2 and an actuator assembly of a delivery apparatus, according to one example.

FIG. 5 is a perspective view of the actuator and actuator assembly of FIG. 4 with the actuator assembly physically coupled to the actuator.

FIG. 6 is a side elevation view of a portion of an example of a frame and an actuation member operatively coupled to a vertical post of the frame.

FIG. 7 is a side elevation view of a portion of an example of a frame and an actuation member operatively coupled to a vertical post of the frame, with the frame shown in a radially expanded configuration.

FIG. 8 is an enlarged view of a portion of the actuation member and vertical post of the frame of FIG. 7.

FIG. 9 is a side elevation view of the frame portion of FIG. 7 shown in a radially compressed configuration.

### DETAILED DESCRIPTION

### General Considerations

For purposes of this description, certain aspects, advantages, and novel features of examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present, or problems be solved.

Although the operations of some of the disclosed examples are described in a particular, sequential order for convenient presentation, it should be understood that this manner of description encompasses rearrangement, unless a particular ordering is required by specific language set forth herein. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (for example, out of the patient's body), while distal motion of the device is motion of the device away from the user and toward the implantation site (for example, into the patient's body). The terms “longitudinal” and “axial” refer to an axis extending in the proximal and distal directions, unless otherwise expressly defined.

### Introduction to the Disclosed Technology

Prosthetic valves disclosed herein can be radially compressible and expandable between a radially compressed state and a radially expanded state. Thus, the prosthetic valves can be crimped on or retained by an implant delivery apparatus in the radially compressed state while being advanced through a patient's vasculature on the delivery apparatus. The prosthetic valve can be expanded to the radially expanded state once the prosthetic valve reaches the implantation site. It is understood that the prosthetic valves disclosed herein may be used with a variety of implant delivery apparatuses and can be implanted via various delivery procedures, examples of which will be discussed in more detail later.

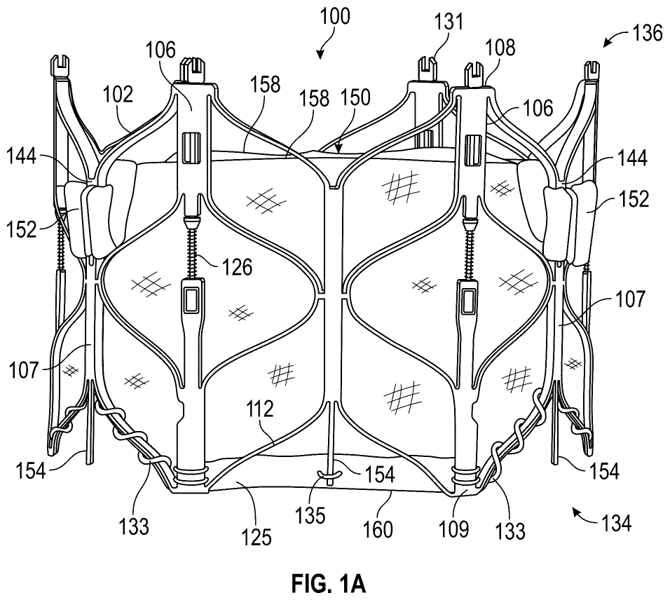

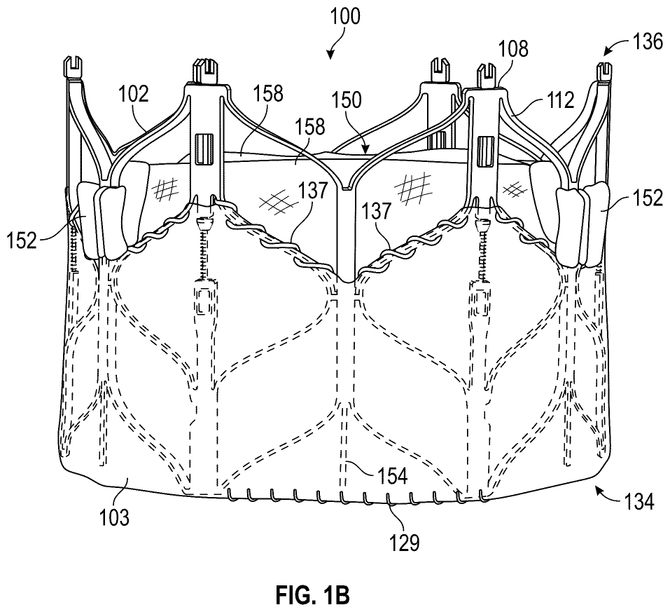

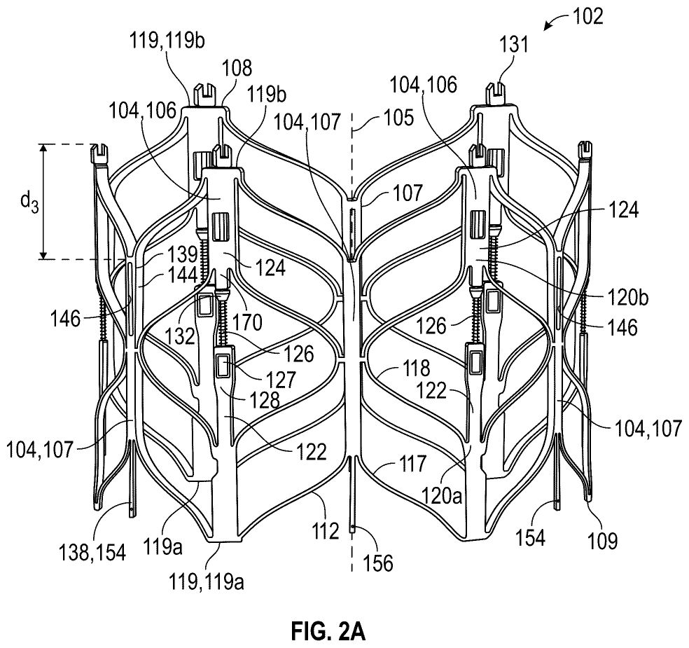

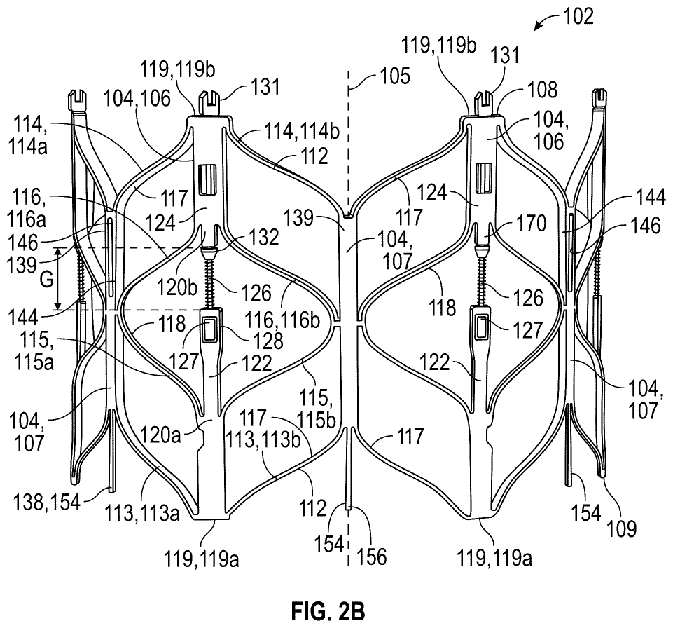

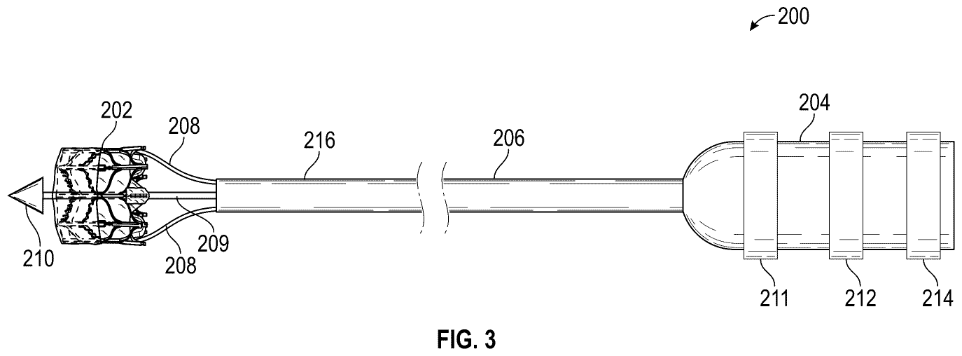

FIGS. 1A-2B illustrate an exemplary prosthetic device (for example, prosthetic heart valve) that can be advanced through a patient's vasculature, such as to a native heart valve, by a delivery apparatus, such as the exemplary delivery apparatus shown in FIG. 3. The frame of the prosthetic heart valve can include one or more mechanical expansion and locking mechanisms that can be integrated into the frame—specifically, into axially extending posts of the frame. The mechanical expansion and/or locking mechanisms can be removably coupled to, and/or actuated by, the delivery apparatus to radially expand the prosthetic heart valve and lock the prosthetic heart valve in one or more radially expanded states.

### Examples of the Disclosed Technology

FIGS. 1A-2B show an exemplary prosthetic valve 100, according to one example. Any of the prosthetic valves disclosed herein are adapted to be implanted in the native aortic annulus, although in some examples they can be adapted to be implanted in the other native annuluses of the heart (the pulmonary, mitral, and tricuspid valves). The disclosed prosthetic valves also can be implanted within vessels communicating with the heart, including a pulmonary artery (for replacing the function of a diseased pulmonary valve, or the superior vena cava or the inferior vena cava (for replacing the function of a diseased tricuspid valve) or various other veins, arteries and vessels of a patient. The disclosed prosthetic valves also can be implanted within a previously implanted prosthetic valve (which can be a prosthetic surgical valve or a prosthetic transcatheter heart valve) in a valve-in-valve procedure.

In some examples, the disclosed prosthetic valves can be implanted within a docking or anchoring device that is implanted within a native heart valve or a vessel. For example, in one example, the disclosed prosthetic valves can be implanted within a docking device implanted within the pulmonary artery for replacing the function of a diseased pulmonary valve, such as disclosed in U.S. Publication No. 2017/0231756, which is incorporated by reference herein. In an example, the disclosed prosthetic valves can be implanted within a docking device implanted within or at the native mitral valve, such as disclosed in PCT Publication No. WO2020/247907, which is incorporated herein by reference. In an example, the disclosed prosthetic valves can be implanted within a docking device implanted within the superior or inferior vena cava for replacing the function of a diseased tricuspid valve, such as disclosed in U.S. Publication No. 2019/0000615, which is incorporated herein by reference.

FIGS. 1A-2B illustrate an example of a prosthetic valve 100 (which also may be referred to herein as “prosthetic heart valve 100”) having a frame 102. FIGS. 2A-2B show the frame 102 by itself, while FIGS. 1A-1B show the frame 102 with a valvular structure 150 (which can comprise leaflets 158, as described further herein) mounted within and to the annular frame 102. FIG. 1B additionally shows an optional skirt assembly comprising an outer skirt 103. While only one side of the frame 102 is depicted in FIG. 2B, it should be appreciated that the frame 102 forms an annular structure having an opposite side that is substantially identical to the portion shown in FIG. 1B, as shown in FIGS. 1A-2A.

As shown in FIGS. 1A and 1B, the valvular structure 150 is coupled to and supported inside the frame 102. The valvular structure 150 is configured to regulate the flow of blood through the prosthetic valve 100, from an inflow end portion 134 to an outflow end portion 136. The valvular structure 150 can include, for example, a leaflet assembly comprising one or more leaflets 158 made of flexible material. The leaflets 158 can be made from in whole or part, biological material, bio-compatible synthetic materials, or other such materials. Suitable biological material can include, for example, bovine pericardium (or pericardium from other sources). The leaflets 158 can be secured to one another at their adjacent sides to form commissures 152, each of which can be secured to a respective commissure support structure 144 (also referred to herein as “commissure supports”) and/or to other portions of the frame 102, as described in greater detail herein.

In the example depicted in FIGS. 1A and 1B, the valvular structure 150 includes three leaflets 158, which can be arranged to collapse in a tricuspid arrangement. Each leaflet 158 can have an inflow edge portion 160 (which can also be referred to as a cusp edge portion) (FIG. 1A). The inflow edge portions 160 of the leaflets 158 can define an undulating, curved scallop edge that generally follows or tracks portions of struts 112 of frame 102 in a circumferential direction when the frame 102 is in the radially expanded configuration. The inflow edge portions 160 of the leaflets 158 can be referred to as a “scallop line.”

The prosthetic valve 100 may include one or more skirts mounted around the frame 102. For example, as shown in FIG. 1B, the prosthetic valve 100 may include an outer skirt 103 mounted around an outer surface of the frame 102. The outer skirt 103 can function as a sealing member for the prosthetic valve 100 by sealing against the tissue of the native valve annulus and helping to reduce paravalvular leakage past the prosthetic valve 100. In some cases, an inner skirt (not shown) may be mounted around an inner surface of the frame 102. The inner skirt can function as a sealing member to prevent or decrease perivalvular leakage, to anchor the leaflets 158 to the frame 102, and/or to protect the leaflets 158 against damage caused by contact with the frame 102 during crimping and during working cycles of the prosthetic valve 100. In some examples, the inflow edge portions 160 of the leaflets 158 can be sutured to the inner skirt generally along the scallop line. The inner skirt can in turn be sutured to adjacent struts 112 of the frame 102. In some examples, as shown in FIG. 1A, the leaflets 158 can be sutured directly to the frame 102 or to a reinforcing member 125 (also referred to as a reinforcing skirt or connecting skirt) in the form of a strip of material (for example, a fabric strip) which is then sutured to the frame 102, along the scallop line via stitches (for example, whip stitches) 133.

The inner and outer skirts and the connecting skirt 125 can be wholly or partly formed of any suitable biological material, synthetic material (for example, any of various polymers), or combinations thereof. In some examples, the skirt can comprise a fabric having interlaced yarns or fibers, such as in the form of a woven, braided, or knitted fabric. In some examples, the fabric can have a plush nap or pile. Exemplary fabrics having a plus nap or pile include velour, velvet, velveteen, corduroy, terrycloth, fleece, etc. In some examples, the skirt can comprise a fabric without interlaced yarns or fibers, such as felt or an electrospun fabric. Exemplary materials that can be used for forming such fabrics (with or without interlaced yarns or fibers) include, without limitation, polyethylene (PET), ultra-high molecular weight polyethylene (UHMWPE), polytetrafluoroethylene (PTFE), expanded polytetrafluoroethylene (ePTFE), polyamide etc. In some examples, the skirt can comprise a non-textile or non-fabric material, such as a film made from any of a variety of polymeric materials, such as PTFE, PET, polypropylene, polyamide, polyetheretherketone (PEEK), polyurethane (such as thermoplastic polyurethane (TPU)), etc. In some examples, the skirt can comprise a sponge material or foam, such as polyurethane foam. In some examples, the skirt can comprise natural tissue, such as pericardium (for example, bovine pericardium, porcine pericardium, equine pericardium, or pericardium from other sources).

Further details regarding the assembly of the leaflet assembly and the assembly of the leaflets and the skirts to the frame can be found, for example, in U.S. Provisional Application Nos. 63/209,904, filed Jun. 11, 2021, and 63/224,534, filed Jul. 22, 2021, which are incorporated herein by reference. Further details of the construction and function of the frame 102 can be found in International Patent Application No. PCT/US2021/052745, filed Sep. 30, 2021, which is incorporated herein by reference.

The frame 102, which is shown alone and in greater detail in FIGS. 2A and 2B, comprises and inflow end 109, an outflow end 108, and a plurality of axially extending posts 104. The axial direction of the frame 102 is indicated by a longitudinal axis 105, which extends from the inflow end 109 to the outflow end 108 (FIGS. 2A and 2B). Some of the posts 104 can be arranged in pairs of axially aligned first and second struts or posts 122, 124. An actuator 126 (such as the illustrated threaded rod or bolt) can extend through one or more pairs of posts 122, 124 to form an integral expansion and locking mechanism or actuator mechanism 106 configured to radially expand and compress the frame 102, as further described herein. One or more of posts 104 can be configured as support posts 107.

The frame 102 can be made of any of various suitable plastically-expandable materials (for example, stainless steel, etc.) or self-expanding materials (for example, Nitinol) as known in the art. When constructed of a plastically-expandable material, the frame 102 (and thus the valve 100) can be crimped to a radially compressed state on a delivery catheter and then expanded inside a patient by an inflatable balloon or equivalent expansion mechanism. When constructed of a self-expandable material, the frame 102 (and thus the valve 100) can be crimped to a radially compressed state and restrained in the compressed state by insertion into a sheath or equivalent mechanism of a delivery catheter. Once inside the body, the valve can be advanced from the delivery sheath, which allows the valve to expand to its functional size.

Suitable plastically-expandable materials that can be used to form the frames disclosed herein (for example, the frame 102) include, metal alloys, polymers, or combinations thereof. Example metal alloys can comprise one or more of the following: nickel, cobalt, chromium, molybdenum, titanium, or other biocompatible metal. In some examples, the frame 102 can comprise stainless steel. In some examples, the frame 102 can comprise cobalt-chromium. In some examples, the frame 102 can comprise nickel-cobalt-chromium. In some examples, the frame 102 comprises a nickel-cobalt-chromium-molybdenum alloy, such as MP35N™ (tradename of SPS Technologies), which is equivalent to UNS R30035 (covered by ASTM F562-02). MP35N™/UNS R30035 comprises 35% nickel, 35% cobalt, 20% chromium, and 10% molybdenum, by weight.

The actuator mechanisms 106 (which can be used to radially expand and/or radially compress the prosthetic valve 100) can be integrated into the frame 102 of the prosthetic valve 100, thereby reducing the crimp profile and/or bulk of the prosthetic valve 100. Integrating the actuator mechanisms 106 (which can also be referred to herein as “expansion and locking mechanisms”) into the frame 102 can also simplify the design of the prosthetic valve 100, making the prosthetic valve 100 less costly and/or easier to manufacture. In the illustrated example, an actuator 126 extends through each pair of axially aligned posts 122, 124. In some examples, one or more of the pairs of posts 122, 124 can be without a corresponding actuator.

The posts 104 can be coupled together by a plurality of circumferentially extending link members or struts 112. Each strut 112 extends circumferentially between adjacent posts 104 to connect all of the axially extending posts 104. As an example, the prosthetic valve 100 can include equal numbers of support posts 107 and pairs of actuator posts 122, 124 and the pairs of posts 122, 124 and the support posts 107 can be arranged in an alternating order such that each strut 112 is positioned between one of the pairs of posts 122, 124 and one of the support posts 107 (that is, each strut 112 can be coupled on one end to one of the posts 122, 124 and can be coupled on the other end to one of the support posts 107). However, the prosthetic valve 100 can include different numbers of support posts 107 and pairs of posts 122, 124 and/or the pairs of posts 122, 124 and the support posts 107 can be arranged in a non-alternating order, in some examples.

As illustrated in FIG. 2B, the struts 112 can include a first row of struts 113 at or near the inflow end 109 of the prosthetic valve 100, a second row of struts 114 at or near the outflow end 108 of the prosthetic valve 100, and third and fourth rows of struts 115, 116, respectively, positioned axially between the first and second rows of struts 113, 114. The struts 112 can form and/or define a plurality of cells (that is, openings) in the frame 102. For example, the struts 113, 114, 115, and 116 can at least partially form and/or define a plurality of first cells 117 and a plurality of second cells 118 that extend circumferentially around the frame 102. Specifically, each first cell 117 can be formed by two struts 113a, 113b of the first row of struts 113, two struts 114a, 114b of the second row of struts 114, and two of the support posts 107. Each second cell 118 can be formed by two struts 115a, 115b of the third row of struts 115 and two struts 116a, 116b of the fourth row of struts 116. As illustrated in FIGS. 2A and 2B, each second cell 118 can be disposed within one of the first cells 117 (that is, the struts 115a-116b forming the second cells 118 are disposed between the struts forming the first cells 117 (that is, the struts 113a, 113b and the struts 114a, 114b), closer to an axial midline of the frame 102 than the struts 113a-114b).

As illustrated in FIGS. 2A and 2B, the struts 112 of frame 102 can comprise a curved shape. Each first cell 117 can have an axially-extending hexagonal shape including first and second apices 119 (for example, an inflow apex 119a and an outflow apex 119b). In examples where the delivery apparatus is releasably connected to the outflow apices 119b (as described herein), each inflow apex 119a can be referred to as a “distal apex” and each outflow apex 119b can be referred to as a “proximal apex”. Each second cell 118 can have a diamond shape including first and second apices 120 (for example, distal apex 120a and proximal apex 120b). In some examples, the frame 102 comprises six first cells 117 extending circumferentially in a row, six second cells 118 extending circumferentially in a row within the six first cells 117, and twelve posts 104. In some examples, the frame 102 can comprise a greater or fewer number of first cells 117 and a correspondingly greater or fewer number of second cells 118 and posts 104.

As noted herein, some of the posts 104 can be arranged in pairs of first and second posts 122, 124. The posts 122, 124 are aligned with each other along the length of the frame 102 and are axially separated from one another by a gap G (FIG. 2B) (those with actuators 126 can be referred to as actuator posts or actuator struts). Each first post 122 (that is, the lower post shown in FIGS. 2A and 2B) can extend axially from the inflow end 109 of the prosthetic valve 100 toward the second post 124, and the second post 124 (that is, the upper post shown in FIGS. 2A and 2B) can extend axially from the outflow end 108 of the prosthetic valve 100 toward the first post 122. For example, each first post 122 can be connected to and extend from an inflow apex 119a and each second post 124 can be connected to and extend from an outflow apex 119b. Each first post 122 and the second post 124 can include an inner bore configured to receive a portion of an actuator member, such as in the form of a substantially straight threaded rod 126 (or bolt) as shown in the illustrated example. The threaded rod 126 also may be referred to herein as actuator 126, actuator member 126, and/or screw actuator 126. In examples where the delivery apparatus can be releasably connected to the outflow end 108 of the frame 102, the first posts 122 can be referred to as distal posts or distal axial struts and the second posts 124 can be referred to as proximal posts or proximal axial struts.

Each threaded rod 126 extends axially through a corresponding first post 122 and second post 124. Each threaded rod 126 also extends through a bore of a nut 127 captured within a slot or window formed in an end portion 128 of the first post 122. The threaded rod 126 has external threads that engage internal threads of the bore of the nut 127. The inner bore of the second post 124 (through which the threaded rod 126 extends) can have a smooth and/or non-threaded inner surface to allow the threaded rod 126 to slide freely within the bore. Rotation of the threaded rod 126 relative to the nut 127 produces radial expansion and compression of the frame 102, as further described herein.

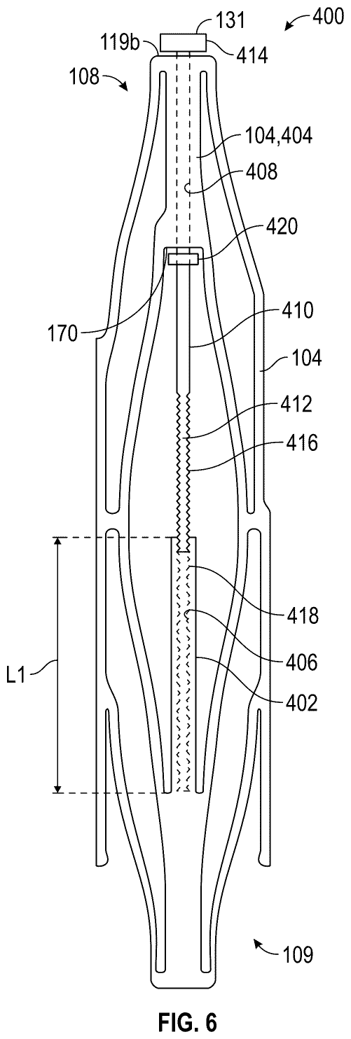

In some examples, the threaded rod 126 can extend past the nut 127 toward the inflow end 109 of the frame 102 into the inner bore of the first post 122. The nut 127 can be held in a fixed position relative to the first post 122 such that the nut 127 does not rotate relative to the first post 122. In this way, whenever the threaded rod 126 is rotated (for example, by a physician) the threaded rod 126 can rotate relative to both the nut 127 and the first post 122. The engagement of the external threads of the threaded rod 126 and the internal threads of the nut 127 prevent the rod 126 from moving axially relative to the nut 127 and the first post 122 unless the threaded rod 126 is rotated relative to the nut 127. Thus, the threaded rod 126 can be retained or held by the nut 127 and can only be moved relative to the nut 127 and/or the first post 122 by rotating the threaded rod 126 relative to the nut 127 and/or the first post 122. In some examples, in lieu of using the nut 127, at least a portion of the inner bore of the first post 122 can be threaded. For example, the bore along the end portion 128 of the first post 122 can comprise inner threads that engage the external threaded rod 126 such that rotation of the threaded rod causes the threaded rod 126 to move axially relative to the first post 122 (as shown in FIG. 6 and described in greater detail herein).

When a threaded rod 126 extends through and/or is otherwise coupled to a pair of axially aligned posts 122, 124, the pair of axially aligned posts 122, 124 and the threaded rod 126 can serve as one of the expansion and locking mechanisms 106. In some examples, a threaded rod 126 can extend through each pair of axially aligned posts 122, 124 so that all of the posts 122, 124 (with their corresponding rods 126) serve as expansion and locking mechanisms 106. As just one example, the prosthetic valve 100 can include six pairs of posts 122, 124, and each of the six pairs of posts 122, 124 with their corresponding rods 126 can be configured as one of the expansion and locking mechanisms 106 for a total of six expansion and locking mechanisms 106. In some examples, not all pairs of posts 122, 124 need be expansion and locking mechanisms (that is, actuators). If a pair of posts 122, 124 is not used as an expansion and locking mechanism, a threaded rod 126 need not extend through the posts 122, 124 of that pair.

The threaded rod 126 can be rotated relative to the nut 127, the first post 122, and the second post 124 to axially foreshorten and/or axially elongate the frame 102, thereby radially expanding and/or radially compressing, respectively, the frame 102 (and therefore the prosthetic valve 100). Specifically, when the threaded rod 126 is rotated relative to the nut 127, the first post 122, and the second post 124, the first and second posts 122, 124 can move axially relative to one another, thereby widening or narrowing the gap G (FIG. 2B) separating the posts 122, 124, and thereby radially compressing or radially expanding the prosthetic valve 100, respectively. Thus, the gap G (FIG. 2B) between the first and second posts 122, 124 narrows as the frame 102 is radially expanded and widens as the frame 102 is radially compressed.

The threaded rod 126 can extend proximally past the proximal end of the second post 124 and can include a head portion 131 at its proximal end that can serve at least two functions. First, the head portion 131 can removably or releasably couple the threaded rod 126 to a respective actuator assembly of a delivery apparatus that can be used to radially expand and/or radially compress the prosthetic valve 100 (for example, the delivery apparatus 200 of FIG. 3, as described herein). Second, the head portion 131 can prevent the second post 124 from moving proximally relative to the threaded rod 126 and can apply a distally directed force to the second post 124, such as when radially expanding the prosthetic valve 100. Specifically, the head portion 131 can have a width greater than a diameter of the inner bore of the second post 124 such that the head portion 131 is prevented from moving into the inner bore of the second post 124. Thus, as the threaded rod 126 is threaded farther into the nut 127, the head portion 131 of the threaded rod 126 draws closer to the nut 127 and the first post 122, thereby drawing the second post 124 towards the first post 122, and thereby axially foreshortening and radially expanding the prosthetic valve 100.

The threaded rod 126 also can include a stopper 132 (for example, in the form of a nut, washer or flange) disposed thereon. The stopper 132 can be disposed on the threaded rod 126 such that it sits within the gap G. Further, the stopper 132 can be integrally formed on or fixedly coupled to the threaded rod 126 such that it does not move relative to the threaded rod 126. Thus, the stopper 132 can remain in a fixed axial position on the threaded rod 126 such that it moves in lockstep with the threaded rod 126.

Rotation of the threaded rod 126 in a first direction (for example, clockwise) can cause corresponding axial movement of the first and second posts 122, 124 toward one another, thereby decreasing the gap G and radially expanding the frame 102, while rotation of the threaded rod 126 in an opposite second direction causes corresponding axial movement of the first and second posts 122, 124 away from one another, thereby increasing the gap G and radially compressing the frame. When the threaded rod 126 is rotated in the first direction, the head portion 131 of the rod 126 bears against an adjacent surface of the frame (for example, an outflow apex 119b), while the nut 127 and the first post 122 travel proximally along the threaded rod 126 toward the second post 124, thereby radially expanding the frame. As the frame 102 moves from a compressed configuration to an expanded configuration, the gap G between the first and second posts 122, 124 can narrow.

When the threaded rod 126 is rotated in the second direction, the threaded rod 126 and the stopper 132 move toward the outflow end 108 of the frame until the stopper 132 abuts the inflow end 170 of the second post 124 (as shown in FIGS. 2A and 2B). Upon further rotation of the rod 126 in the second direction, the stopper 132 can apply a proximally directed force to the second post 124 to radially compress the frame 102. Specifically, during crimping/radial compression of the prosthetic valve 100, the threaded rod 126 can be rotated in the second direction (for example, counterclockwise) causing the stopper 132 to push against (that is, provide a proximally directed force to) the inflow end 170 of the second post 124, thereby causing the second post 124 to move away from the first post 122, and thereby axially elongating and radially compressing the prosthetic valve 100.

Thus, each of the second posts 124 can slide axially relative to a corresponding one of the first posts 122 but can be axially retained and/or restrained between the head portion 131 of a threaded rod 126 and a stopper 132. That is, each second post 124 can be restrained at its proximal end by the head portion 131 of the threaded rod 126 and at its distal end by the stopper 132. In this way, the head portion 131 can apply a distally directed force to the second post 124 to radially expand the prosthetic valve 100 while the stopper 132 can apply a proximally directed force to the second post 124 to radially compress the prosthetic valve 100. As explained herein, radially expanding the prosthetic valve 100 axially foreshortens the prosthetic valve 100, causing an inflow end portion 134 and outflow end portion 136 of the prosthetic valve 100 (FIGS. 1A and 1B) to move towards one another axially, while radially compressing the prosthetic valve 100 axially elongates the prosthetic valve 100, causing the inflow and outflow end portions 134, 136 to move away from one another axially.

In some examples, the threaded rod 126 can be fixed against axial movement relative to the second post 124 (and the stopper 132 can be omitted) such that rotation of the threaded rod 126 in the first direction produces proximal movement of the nut 127 and radial expansion of the frame 102 and rotation of the threaded rod 126 in the second direction produces distal movement of the nut 127 and radial compression of the frame 102.

As also introduced herein, some of the posts 104 can be configured as support posts 107. As shown in FIGS. 2A and 2B, the support posts 107 can extend axially between the inflow and outflow ends 109, 108 of the frame 102 and each can have an inflow end portion 138 and an outflow end portion 139. The outflow end portion 139 of one or more support posts 107 can include a commissure support structure or member 144. The commissure support structure 144 can comprise strut portions defining a commissure opening 146 therein.

The commissure opening 146 (which can also be referred to herein as a “commissure window 146”) can extend radially through a thickness of the support post 107 and can be configured to accept a portion of a valvular structure 150 (for example, a commissure 152) to couple the valvular structure 150 to the frame 102. For example, each commissure 152 can be mounted to a respective commissure support structure 144, such as by inserting a pair of commissure tabs of adjacent leaflets 158 through the commissure opening 146 and suturing the commissure tabs to each other and/or the commissure support structure 144. In some examples, the commissure opening 146 can be fully enclosed by the support post 107 such that a portion of the valvular structure 150 can be slid radially through the commissure opening 146, from an interior to an exterior of the frame 102, during assembly. In the illustrated example, the commissure opening 146 has a substantially rectangular shape that is shaped and sized to receive commissure tabs of two adjacent leaflets therethrough. However, in some examples, the commissure opening can have any of various shapes (for example, square, oval, square-oval, triangular, L-shaped, T-shaped, C-shaped, etc.).

The commissure openings 146 are spaced apart about the circumference of frame 102 (or angularly spaced apart about frame 102). The spacing may or may not be even. In one example, the commissure openings 146 are axially offset from the outflow end 108 of the frame 102 by an offset distance d₃ (indicated in FIG. 2A). As an example, the offset distance d₃ may be in a range from 2 mm to 6 mm. In general, the offset distance d₃ should be selected such that when the leaflets are attached to the frame 102 via the commissure openings 146, the free edge portions (for example, outflow edge portions) of the leaflets 158 will not protrude from or past the outflow end 108 of the frame 102.

The frame 102 can comprise any number of support posts 107, any number of which can be configured as commissure support structures 144. For example, the frame 102 can comprise six support posts 107, three of which are configured as commissure support structures 144. However, in some examples, the frame 102 can comprise more or less than six support posts 107 and/or more or less than three commissure support structures 144.

The inflow end portion 138 of each support post 107 can comprise an extension 154 (show as a cantilevered strut in FIGS. 2A and 2B) that extends toward the inflow end 109 of the frame 102. Each extension 154 can comprise an aperture 156 extending radially through a thickness of the extension 154. In some examples, the extension 154 can extend such that an inflow edge of the extension 154 aligns with or substantially aligns with the inflow end 109 of the frame 102. In use, the extension 154 can prevent or mitigate portions of an outer skirt from extending radially inwardly and thereby prevent or mitigate any obstruction of flow through the frame 102 caused by the outer skirt. The extensions 154 can further serve as supports to which portions of the inner and/or outer skirts and/or the leaflets and/or the connecting skirt 125 can be coupled. For example, sutures used to connect the inner and/or outer skirts and/or the leaflets and/or the connecting skirt 125 can be wrapped around the extensions 154 and/or can extend through apertures 156.

As an example, each extension 154 can have an aperture 156 (FIG. 2A) or other features to receive a suture or other attachment material for connecting an adjacent inflow edge portion 160 of a leaflet 158 (FIG. 1A), the outer skirt 103 (in FIG. 1B), the connecting skirt 125, and/or an inner skirt. In some examples, the inflow edge portion 160 of each leaflet 158 can be connected to a corresponding extension via a suture 135 (FIG. 1A).

In some examples, the outer skirt 103 can be mounted around the outer surface of frame 102 as shown in FIG. 1B and the inflow edge of the outer skirt 103 (lower edge in FIG. 1B) can be attached to the connecting skirt 125 and/or the inflow edge portions 160 of the leaflets 158 that have already been secured to frame 102 as well as to the extensions 154 of the frame by sutures 129. The outflow edge of the outer skirt 103 (the upper edge in FIG. 1B) can be attached to selected struts with stitches 137. In implementations where the prosthetic valve includes an inner skirt, the inflow edge of the inner skirt can be secured to the inflow edge portions 160 before securing the cusp edge portions to the frame so that the inner skirt will be between the leaflets and the inner surface of the frame. After the inner skirt and leaflets are secured in place, then the outer skirt can be mounted around the frame as described herein.

The frame 102 can be a unitary and/or fastener-free frame that can be constructed from a single piece of material (for example, Nitinol, stainless steel or a cobalt-chromium alloy), such as in the form of a tube. The plurality of cells can be formed by removing portions (for example, via laser cutting) of the single piece of material. The threaded rods 126 can be separately formed and then be inserted through the bores in the second (proximal) posts 124 and threaded into the threaded nuts 127.

In some examples, the frame 102 can be formed from a plastically-expandable material, such as stainless steel or a cobalt-chromium alloy. When the frame is formed from a plastically-expandable material, the prosthetic valve 100 can be placed in a radially compressed state along the distal end portion of a delivery apparatus for insertion into a patient's body. When at the desired implantation site, the frame 102 (and therefore the prosthetic valve 100) can be radially expanded from the radially compressed state to a radially expanded state via actuation of actuation assemblies of the delivery apparatus (as further described herein), which rotate the rods 126 to produce expansion of the frame 102. During delivery to the implantation site, the prosthetic valve 100 can be placed inside of a delivery capsule (sheath) to protect against the prosthetic valve contacting the patient's vasculature, such as when the prosthetic valve is advanced through a femoral artery. The capsule can also retain the prosthetic valve in a compressed state having a slightly smaller diameter and crimp profile than may be otherwise possible without a capsule by preventing any recoil (expansion) of the frame once it is crimped onto the delivery apparatus.

In some examples, the frame 102 can be formed from a self-expandable material (for example, Nitinol). When the frame 102 is formed from a self-expandable material, the prosthetic valve can be radially compressed and placed inside the capsule of the delivery apparatus to maintain the prosthetic valve in the radially compressed state while it is being delivered to the implantation site. When at the desired implantation site, the prosthetic valve is deployed or released from the capsule. In some examples, the frame (and therefore the prosthetic valve) can partially self-expand from the radially compressed state to a partially radially expanded state. The frame 102 (and therefore the prosthetic valve 100) can be further radially expanded from the partially expanded state to a further radially expanded state via actuation of actuation assemblies of the delivery apparatus (as further described herein), which rotate the rods 126 to produce expansion of the frame.

As introduced herein, the threaded rods 126 can removably couple the prosthetic valve 100 to actuator assemblies of a delivery apparatus. Referring to FIG. 3, it illustrates an exemplary delivery apparatus 200 for delivering a prosthetic device or valve 202 (for example, prosthetic valve 100) to a desired implantation location. The prosthetic valve 202 can be releasably coupled to the delivery apparatus 200. It should be understood that the delivery apparatus 200 and other delivery apparatuses disclosed herein can be used to implant prosthetic devices other than prosthetic valves, such as stents or grafts.

The delivery apparatus 200 in the illustrated example generally includes a handle 204, a first elongated shaft 206 (which comprises an outer shaft in the illustrated example) extending distally from the handle 204, at least one actuator assembly 208 extending distally through the first shaft 206, a second elongated shaft 209 (which comprises an inner shaft in the illustrated example) extending through the first shaft 206, and a nosecone 210 coupled to a distal end portion of the second shaft 209. The second shaft 209 and the nosecone 210 can define a guidewire lumen for advancing the delivery apparatus through a patient's vasculature over a guidewire. The at least one actuator assembly 208 can be configured to radially expand and/or radially collapse the prosthetic valve 202 when actuated, such as by one or more knobs 211, 212, 214 included on the handle 204 of the delivery apparatus 200.

Though the illustrated example shows two actuator assemblies 208 for purposes of illustration, it should be understood that one actuator assembly 208 can be provided for each actuator (for example, actuator or threaded rod 126) on the prosthetic valve. For example, three actuator assemblies 208 can be provided for a prosthetic valve having three actuators. In some examples, a greater or fewer number of actuator assemblies can be present.

In some examples, a distal end portion 216 of the shaft 206 can be sized to house the prosthetic valve in its radially compressed, delivery state during delivery of the prosthetic valve through the patient's vasculature. In this manner, the distal end portion 216 functions as a delivery sheath or capsule for the prosthetic valve during delivery,

The actuator assemblies 208 can be releasably coupled to the prosthetic valve 202. For example, in the illustrated example, each actuator assembly 208 can be coupled to a respective actuator (for example, threaded rod 126) of the prosthetic valve 202. Each actuator assembly 208 can comprise a support tube and an actuator member. When actuated, the actuator assembly can transmit pushing and/or pulling forces to portions of the prosthetic valve to radially expand and collapse the prosthetic valve as previously described. The actuator assemblies 208 can be at least partially disposed radially within, and extend axially through, one or more lumens of the first shaft 206. For example, the actuator assemblies 208 can extend through a central lumen of the shaft 206 or through separate respective lumens formed in the shaft 206.

The handle 204 of the delivery apparatus 200 can include one or more control mechanisms (for example, knobs or other actuating mechanisms) for controlling different components of the delivery apparatus 200 in order to expand and/or deploy the prosthetic valve 202. For example, in the illustrated example the handle 204 comprises first, second, and third knobs 211, 212, and 214, respectively.

The first knob 211 can be a rotatable knob configured to produce axial movement of the first shaft 206 relative to the prosthetic valve 202 in the distal and/or proximal directions in order to deploy the prosthetic valve from the delivery sheath 216 once the prosthetic valve has been advanced to a location at or adjacent the desired implantation location with the patient's body. For example, rotation of the first knob 211 in a first direction (for example, clockwise) can retract the sheath 216 proximally relative to the prosthetic valve 202 and rotation of the first knob 211 in a second direction (for example, counter-clockwise) can advance the sheath 216 distally. In some examples, the first knob 211 can be actuated by sliding or moving the first knob 211 axially, such as pulling and/or pushing the knob. In some examples, actuation of the first knob 211 (rotation or sliding movement of the first knob 211) can produce axial movement of the actuator assemblies 208 (and therefore the prosthetic valve 202) relative to the delivery sheath 216 to advance the prosthetic valve distally from the sheath 216.

The second knob 212 can be a rotatable knob configured to produce radial expansion and/or compression of the prosthetic valve 202. For example, rotation of the second knob 212 can rotate the threaded rods of the prosthetic valve 202 via the actuator assemblies 208. Rotation of the second knob 212 in a first direction (for example, clockwise) can radially expand the prosthetic valve 202 and rotation of the second knob 212 in a second direction (for example, counter-clockwise) can radially collapse the prosthetic valve 202. In some examples, the second knob 212 can be actuated by sliding or moving the second knob 212 axially, such as pulling and/or pushing the knob.

The third knob 214 can be a rotatable knob operatively connected to a proximal end portion of each actuator assembly 208. The third knob 214 can be configured to retract an outer sleeve or support tube of each actuator assembly 208 to disconnect the actuator assemblies 208 from the proximal portions of the actuators of the prosthetic valve (for example, threaded rod). Once the actuator assemblies 208 are uncoupled from the prosthetic valve 202, the delivery apparatus 200 can be removed from the patient, leaving just the prosthetic valve 202 in the patient.

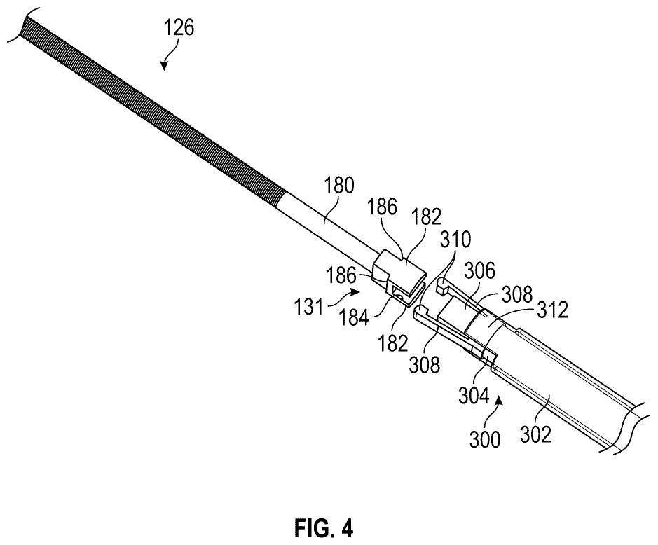

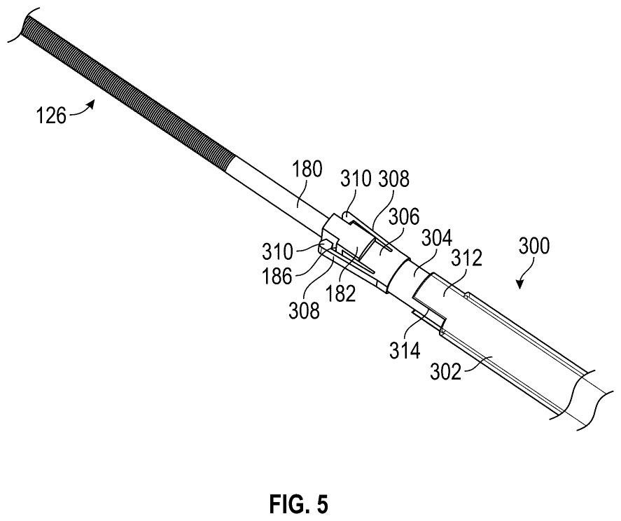

Referring to FIGS. 4-5, they illustrate how each of the threaded rods 126 of the prosthetic device 100 can be removably coupled to an exemplary actuator assembly 300 (for example, actuator assemblies 208) of a delivery apparatus (for example, delivery apparatus 200). Specifically, FIG. 5 illustrates how one of the threaded rods 126 can be coupled to an actuator assembly 300, while FIG. 4 illustrates how the threaded rod 126 can be detached from the actuator assembly 300.

As introduced herein, an actuator assembly 300 can be coupled to the head portion 131 of each threaded rod 126. The head portion 131 can be included at a proximal end portion 180 of the threaded rod 126 and can extend proximally past a proximal end of the second post 124 (FIG. 2A). The head portion 131 can comprise first and second protrusions 182 defining a channel or slot 184 between them, and one or more shoulders 186. As discussed herein, the head portion 131 can have a width greater than a diameter of the inner bore of the second post 124 such that the head portion 131 is prevented from moving into the inner bore of the second post 124 and such that the head portion 131 abuts the outflow end 108 of the frame 102. In particular, the head portion 131 can abut an outflow apex 119b of the frame 102. The head portion 131 can be used to apply a distally-directed force to the second post 124, for example, during radial expansion of the frame 102.

Each actuator assembly 300 can comprise a first actuation member configured as a support tube or outer sleeve 302 and a second actuation member configured as a driver 304. The driver 304 can extend through the outer sleeve 302. The outer sleeve 302 is shown transparently in FIGS. 4-5 for purposes of illustration. The distal end portions of the outer sleeve 302 and driver 304 can be configured to engage or abut the proximal end of the threaded rod 126 (for example, the head portion 131) and/or the frame 102 (for example, the apex 119b). The proximal portions of the outer sleeve 302 and driver 304 can be operatively coupled to the handle of a delivery apparatus (for example, handle 204). The delivery apparatus in this example can include the same features described previously for delivery apparatus 200. In particular examples, the proximal end portions of each driver 304 can be operatively connected to the knob 212 such that rotation of the knob 212 (clockwise or counterclockwise) causes corresponding rotation of the drivers 304. The proximal end portions of each outer sleeve 302 can be operatively connected to the knob 214 such that rotation of the knob 214 (clockwise or counterclockwise) causes corresponding axial movement of the sleeves 302 (proximally or distally) relative to the drivers 304. In some examples, the handle can include electric motors for actuating these components.

The distal end portion of the driver 304 can comprise a central protrusion 306 configured to extend into the slot 184 of the threaded rod 126, and one or more flexible elongated elements or arms 308 including protrusions or teeth 310 configured to be releasably coupled to the shoulders 186 of the threaded rod 126. The protrusions 310 can extend radially inwardly toward a longitudinal axis of the second actuation member 304. As shown in FIGS. 4-5, the elongated elements 308 can be configured to be biased radially outward to an expanded state, for example, by shape setting the elements 308.

As shown in FIG. 5, to couple the actuator assembly 300 to the threaded rod 126, the driver 304 can be positioned such that the central protrusion 306 is disposed within the slot 184 (FIG. 4) and such that the protrusions 310 of the elongated elements 308 are positioned distally to the shoulders 186. As the outer sleeve 302 is advanced (for example, distally) over the driver 304, the sleeve 302 compresses the elongated elements 308 they abut and/or snap over the shoulders 186, thereby coupling the actuator assembly 300 to the threaded rod 126. Thus, the outer sleeve 302 effectively squeezes and locks the elongated elements 308 and the protrusions 310 of the driver 304 into engagement with (that is, over) the shoulders 186 of the threaded rod 126, thereby coupling the driver 304 to the threaded rod 126.

Because the central protrusion 306 of the driver 304 extends into the slot 184 of the threaded rod 126 when the driver 304 and the threaded rod 126 are coupled, the driver 304 and the threaded rod 126 can be rotational locked such that they co-rotate. So coupled, the driver 304 can be rotated (for example, using knob 212 the handle of the delivery apparatus 200) to cause corresponding rotation of the threaded rod 126 to radially expand or radially compress the prosthetic device. The central protrusion 306 can be configured (for example, sized and shaped) such that it is advantageously spaced apart from the inner walls of the outer sleeve 302, such that the central protrusion 306 does not frictionally contact the outer sleeve 302 during rotation. Though in the illustrated example the central protrusion 306 has a substantially rectangular shape in cross-section, in some examples, the protrusion 306 can have any of various shapes, for example, square, triangular, oval, etc. The slot 184 can be correspondingly shaped to receive the protrusion 306.

The outer sleeve 302 can be advanced distally relative to the driver 304 past the elongated elements 308, until the outer sleeve 302 engages the frame 102 (for example, a second post 124 of the frame 102). The distal end portion of the outer sleeve 302 also can comprise first and second support extensions 312 defining gaps or notches 314 between the extensions 312. The support extensions 312 can be oriented such that, when the actuator assembly 300 is coupled to a respective threaded rod 126, the support extensions 312 extend partially over an adjacent end portion (for example, the upper end portion) of one of the second posts 124 on opposite sides of the post 124. The engagement of the support extensions 312 with the frame 102 in this manner can counter-act rotational forces applied to the frame 102 by the rods 126 during expansion of the frame 102. In the absence of a counter-force acting against these rotational forces, the frame can tend to “jerk” or rock in the direction of rotation of the rods when they are actuated to expand the frame. The illustrated configuration is advantageous in that outer sleeves, when engaging the proximal posts 124 of the frame 102, can prevent or mitigate such jerking or rocking motion of the frame 102 when the frame 102 is radially expanded.

To decouple the actuator assembly 300 from the prosthetic device 100, the sleeve 302 can be withdrawn proximally relative to the driver 304 until the sleeve 302 no longer covers the elongated elements 308 of the driver 304. As described herein, the sleeve 302 can be used to hold the elongated elements 308 against the shoulders 186 of the threaded rod 126 since the elongated elements 308 can be naturally biased to a radial outward position where the elongated elements 308 do not engage the shoulders 186 of the threaded rod 126. Thus, when the sleeve 302 is withdrawn such that it no longer covers/constrains the elongated elements 308, the elongated elements 308 can naturally and/or passively deflect away from, and thereby release from, the shoulders 186 of the threaded rod 126, thereby decoupling the driver 304 from the threaded rod 126.

The sleeve 302 can be advanced (moved distally) and/or retracted (moved proximally) relative to the driver 304 via a control mechanism (for example, knob 214) on the handle 204 of the delivery apparatus 200, by an electric motor, and/or by another suitable actuation mechanism. For example, the physician can turn the knob 214 in a first direction to apply a distally directed force to the sleeve 302 and can turn the knob 214 in an opposite second direction to apply a proximally directed force to the sleeve 302. Thus, when the sleeve 302 does not abut the prosthetic device and the physician rotates the knob 214 in the first direction, the sleeve 302 can move distally relative to the driver 304, thereby advancing the sleeve 302 over the driver 304. When the sleeve 302 does abut the prosthetic device, the physician can rotate the knob 214 in the first direction to push the entire prosthetic device distally via the sleeve 302. Further, when the physician rotates the knob 214 in the second direction the sleeve 302 can move proximally relative to the driver 304, thereby withdrawing/retracting the sleeve 302 from the driver 304.

Also disclosed herein are examples of prosthetic heart valve frames having actuation assemblies directly connected to one or more struts of a prosthetic heart valve frame, for example by a threaded connection. In some examples, the actuation assemblies can include a bolt or rod with an internally threaded channel (sometimes called a bore) that receives a corresponding externally threaded end section of a vertically oriented frame post. In some examples, the actuation assemblies can include a bolt or rod with an externally threaded end portion that is received by a corresponding internally threaded channel or bore in a vertically oriented frame post.

Such actuation assemblies provide several advantages over those previously described. For example, because the actuation assemblies are directly threadedly connected to a vertically oriented frame post, there is no need to include an actuator nut to enable the radial expansion and compression of the frame, and therefore no need to widen the vertically oriented frame post to add a window to receive and contain the nut. This can reduce the overall crimp profile of the prosthetic heart valve, due to the smaller vertically oriented frame posts, and reduces the manufacturing complexity of the frame. Furthermore, removing the nut and nut window may improve frame durability by eliminating the wear due to relative motion between the nut and nut window.

FIG. 6 shows a section of an exemplary prosthetic heart valve frame 400 having an actuator with an externally threaded end portion. As shown in FIG. 6, the prosthetic heart valve frame 400 can have the same or substantially the same configuration as the frame 102, as previously described, and can function in the same way as described for the frame 102, except for the differences described herein. A prosthetic heart valve can comprise the frame 400 and any of the components described herein for the prosthetic heart valve 100 (for example, leaflets 158, inner and/or outer skirts, connecting member 125, etc.)

The frame 400 comprises an inflow end 109, an outflow end 108, and a plurality of axially extending posts 104. The axial direction of the frame 400 is indicated by a longitudinal axis, which extends from the inflow end 109 to the outflow end 108 similar to the axis 105 illustrated in FIGS. 2A and 2B. Some of the posts 104 can be arranged in pairs of axially aligned first and second struts or posts 402, 404. The posts 402, 404 can include axially extending first and second bores or channels 406, 408 that admit one or more components of an actuation assembly, such as an actuator 410. The actuator 410 (such as the illustrated threaded rod or bolt) can extend through one or more channels 406, 408 to form an integral expansion and locking mechanism or actuator mechanism configured to radially expand and compress the frame 400, as shown in FIG. 6.

With continued reference to FIG. 6 the actuator 410 has a first end portion 412 (sometimes called distal end portion 412) and a second end portion 414 (sometimes called proximal end portion 414). The actuator 410 extends into and through the second bore 408. The first end portion 412 extends into the first bore 406 and has an externally threaded portion 416 comprising external threads that engage internal threads formed along a corresponding internally threaded portion 418 of the first bore 406. The second end portion 414 of the actuator 410 can include a head portion 131, which is configured to be releasably attached to a delivery system actuator assembly, such as actuator assembly 300 discussed herein and shown in FIGS. 4 and 5.

In some examples, a stopper 420 can be positioned on the actuator 410 between the first post 402 and the second post 404. The stopper may be integrally formed or fixedly coupled to the actuator 410, such that it does not move relative to the actuator 410 and thus remains in a fixed axial position of the actuator 410 and moves in lockstep with the actuator 410 as the frame is radially expanded and radially contracted.

While FIG. 6 shows only a portion of frame 400 having an actuator 410 extending axially through the first bore 406 and the second bore 408 of the pair of posts 402, 404, it is to be understood that frame 400 may comprise a plurality of frame sections similar or substantially identical to that shown in FIG. 6, circumferentially arranged to form the frame 400, similar to the frame 102 described herein and illustrated in FIGS. 1A-2B. In some examples, an actuator 410 extends axially through the first bore 406 and second bore 408 of each portion of the frame 400, as shown in FIG. 6. It is to be understood, however, that in some examples one or more of the portions of frame 400 may omit the actuator 410, and optionally may also omit the first bore 406 and the second bore 408. For example, in a frame comprising six substantially identical portions such as that illustrated in FIG. 2A, there may be an actuator extending through the channels 406, 408 in the pairs of vertical posts 402, 404 of all six frame portions, or there may be an actuator extending through the channels 406, 408 in the pairs of vertical posts 402, 404 of a lesser number of the frame portions, such as five, four, three, two, or one frame portions.

Rotation of the actuator 410 in a first direction can cause corresponding axial movement of the first post 402 and the second post 404 toward one another, thereby decreasing the spacing between the first and second posts 402, 404 and radially expanding the frame, while rotation of the actuator 410 in a second direction opposite to the first direction causes corresponding axial movement of the first and second posts 402, 404 away from one another, thereby increasing the spacing between them and radially compressing the frame.

When the actuator 410 is rotated in the first direction, the head portion 131 of the actuator 410 bears against an adjacent surface of the frame (for example, an outflow apex 119b), while the first post 402 travels proximally along the actuator towards the second post 404, thereby radially expanding the frame 400. As the frame 400 moves from a compressed configuration to an expanded configuration, the gap between the first post 402 and the second post 404 can narrow.

When the actuator 410 is rotated in the second direction, the actuator 410 and the stopper 420 move toward the outflow end 108 of the frame until the stopper 420 abuts the inflow end 170 of the second post 404, in a fashion identical to that described herein, and illustrated in FIGS. 2A and 2B. Upon further rotation of the actuator 410 in the second direction, the stopper 420 can apply a proximally directed force to the second post 204 to radially compress the frame 400. Specifically, during crimping/radial compression of the frame 400, the actuator 410 can be rotated in the second direction causing the stopper 420 to push against the inflow end 170 of the second post 404, thereby causing the second post 404 to move away from the first post 402 and elongating and radially compressing the frame 400.

The range of relative axial motion between the first post 402 and the second post 404 is defined by an axial length L1 of the first bore 406 in the first post 402, as shown in FIG. 6. As such, in some examples, the length L1 of the first bore 406 can be varied to accommodate different desired ranges of radial expansion and compression of the frame 400.

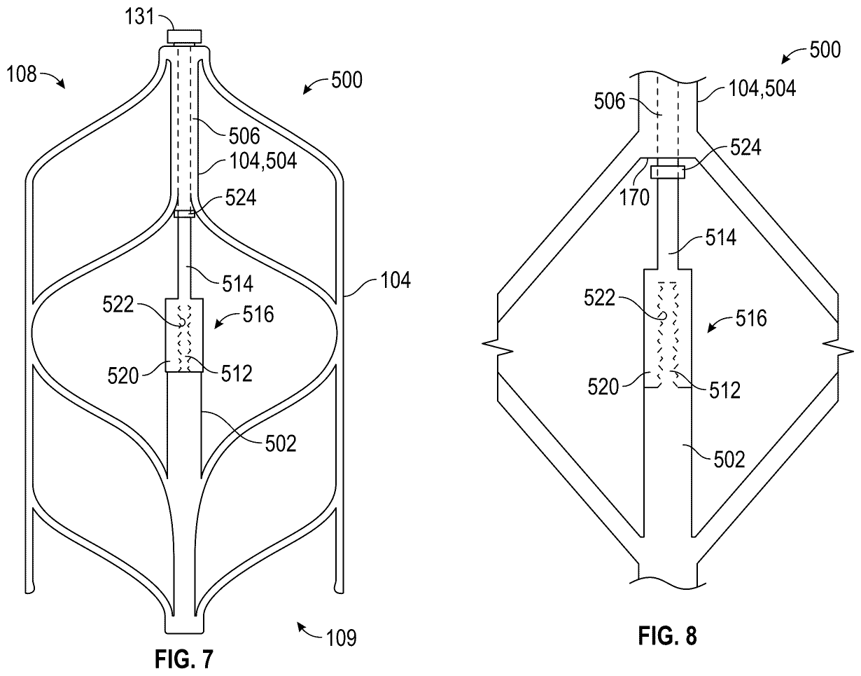

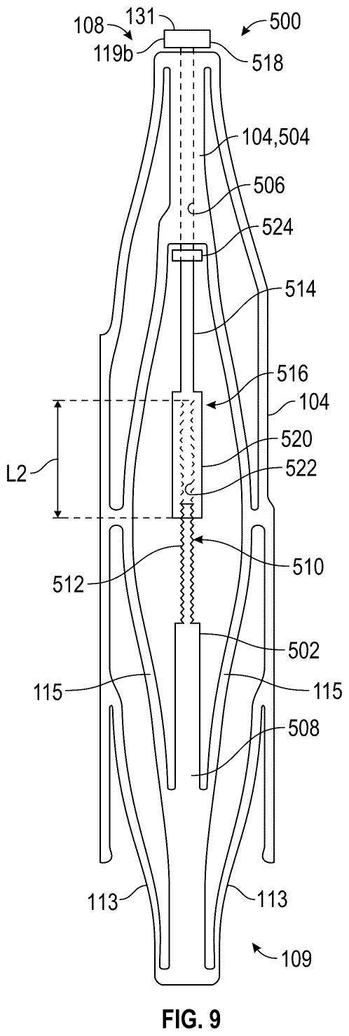

An example prosthetic heart valve frame 500 having an actuation assembly directly connected to one or more struts of the frame is shown in FIGS. 7-9. As shown in FIG. 9, the prosthetic heart valve frame 500 can have the same or substantially the same configuration as the frames 102 and 400, as previously described, and can function in the same way as previously described, except for the differences described herein. A prosthetic heart valve can comprise the frame 500 and any of the components described herein for the prosthetic heart valve 100 (for example, leaflets 158, inner and/or outer skirts, connecting member 125, etc.)

As shown in FIG. 9, the frame 500 can include a plurality of axially extending posts 104. Some of the posts 104 can be arranged in axially aligned pairs of first and second posts 502, 504. The second post 504 can include a channel 506 through which the actuator 514 extends. The first post 502 can comprise a fixed end portion 508 (sometimes called a distal end portion 508) and a free end portion 510 (sometimes called a proximal end portion 510). The fixed end portion 508 can be attached to one or more of the components of the frame 500, such as to pairs of struts 113, 115, as shown in FIG. 9. The free end portion 510 can further include an externally threaded rod 512, which is operatively coupled to the actuator 514.

With continued reference to FIG. 9, the actuator 514 has a first end portion 516 (sometimes called distal end 516) and a second end portion 518 (sometimes called proximal end portion 518). The first end portion 516 can further comprise a cup structure 520 having an internally threaded channel 522 extending at least partially axially through a portion of the cup structure. The internally threaded channel 522 receives the corresponding externally threaded rod 512, and allows the actuator 514 to be rotated in a first rotational direction and a second rotational direction relative to the first post 502. The second end portion 518 of the actuator 514 extends through the channel 506 and can include an enlarged head portion 131 disposed outside of the channel. The head portion 131 is configured to be releasably attached to a delivery system actuator assembly, such as actuator assembly 300, discussed herein and shown in FIGS. 4 and 5.

As shown, the cup structure 520 can have a diameter that is greater than the portion of the actuator 514 extending between the cup structure 520 and the head portion 131. In some examples, the actuator 514 can be formed with an internally threaded bore 522 and, except for the head portion 131, can have a constant diameter along its length.

In some examples, a stopper 524 can be positioned on the actuator 516 between the first post 502 and the second post 504. The stopper may be integrally formed or fixedly coupled to the actuator 516, such that it does not move relative to the actuator 516 and thus remains in a fixed axial position on the actuator 516 and moves in lockstep with the actuator 516 as the frame is radially expanded and radially contracted.

While FIG. 9 shows only a portion of frame 500 having an actuator 516 operatively coupled to components of the frame 500 it is to be understood that in some examples, frame 500 may comprise a plurality of frame sections identical or substantially identical to that shown in FIG. 9, circumferentially arranged to form the frame 500, similar to the frame 102 as described herein and illustrated in FIGS. 1A-2B. In some examples, an actuator 516 extends axially through the bore 506 of each post 504 of the frame 500. It is to be understood however, that in some examples, one or more of the portions of frame 500 may omit the actuator 516, and optionally may also omit the bore 506. For example, in a frame comprising six substantially identical portions such as that illustrated in FIG. 2A, there may be an actuator extending through the channel 506 in the second vertical post 504 of all six frame portions, or there may be an actuator extending through the channel 506 in the second vertical post 504 of a lesser number of frame portions, such as five, four, three, two, or one frame portions.

Rotation of the actuator 516 in a first direction can cause corresponding axial movement of the first post 502 and the second post 504 toward one another, thereby decreasing the spacing between the first and second posts 502, 504 and radially expanding the frame, while rotation of the actuator 516 in a second direction opposite to the first direction causes corresponding axial movement of the first and second posts 502, 504 away from one another, thereby increasing the spacing between them and radially compressing the frame.

When the frame 500 is in the radially compressed configuration, as illustrated in FIG. 9, or in a partially radially compressed configuration, the actuator 516 can be rotated in the first direction, such that the head portion 131 of the actuator 516 bears against an adjacent surface of the frame (for example, an outflow apex 119b), while the first post 502 travels proximally along the actuator towards the second post 504, thereby radially expanding the frame 500. As the frame 500 moves from a compressed configuration to an expanded configuration (that is, from the configuration shown in FIG. 9 to that shown in FIGS. 7 and 8), the gap between the first post 502 and the second post 504 can narrow.

When the frame 500 is in the radially expanded configuration, as illustrated in FIGS. 7 and 8, or in a partially radially compressed configuration, the actuator 516 can be rotated in the second direction, such that the actuator 516 and the stopper 524 move toward the outflow end 108 of the frame 500 until the stopper 524 abuts the inflow end 170 of the second post 504, in a fashion identical to that described herein, and illustrated in FIGS. 2A and 2B. Upon further rotation of the actuator 516 in the second direction, the stopper 524 can apply a proximally directed force to the second post 504 to radially compress the frame 500. Specifically, during crimping/radial compression of the frame 500, the actuator 516 can be rotated in the second direction causing the stopper 526 to push against the inflow end 170 of the second post 504, thereby causing the second post 504 to move away from the first post 502 and elongating and radially compressing the frame 500 (that is, from the configuration shown in FIGS. 7 and 8 to that shown in FIG. 9).

The range of relative axial motion between the first post 502 and the second post 504 is defined by the axial length L2 of the internal bore in the first end of the actuator 516, as shown in FIG. 9. As such, in some examples, the length L2 of the internal can be varied to accommodate different desired ranges of radial expansion and compression of the frame 500.

In this way, the examples previously discussed may provide frames suitable for use with prosthetic heart valves similar to those described previously, which may omit an actuator nut and corresponding window. Such examples may allow for prosthetic heart valves with a narrower crimp profile, and which are not subject to the wear that may arise from the relative motion between an actuator nut and the surrounding components of the frame. Moreover, since fewer components are required, such examples can simplify the manufacturing process for the prosthetic valve.

In some examples, rotatable actuators having internally threaded bores (for example, actuator 514) can be implanted in expandable frames for prosthetic devices (for example, prosthetic valves), in which the frames comprise a plurality of inner and outer struts pivotably connected to each other at a plurality of pivot joints or hinges. Examples of such frames are disclosed in U.S. Publication Nos. 2018/0153689 and 2018/0344456, which are incorporated herein by references. In such examples, one or more pairs of rotatable actuators with internal threads and stationary posts with external threads can be mounted to an inner surface of a frame and/or to an outer surface of a frame.

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples of heat/thermal sterilization include steam sterilization and autoclaving. Examples of radiation for use in sterilization include, without limitation, gamma radiation, ultra-violet radiation, and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

### Delivery Techniques

For implanting a prosthetic valve within the native aortic valve via a transfemoral delivery approach, the prosthetic valve is mounted in a radially compressed state along the distal end portion of a delivery apparatus. The prosthetic valve and the distal end portion of the delivery apparatus are inserted into a femoral artery and are advanced into and through the descending aorta, around the aortic arch, and through the ascending aorta. The prosthetic valve is positioned within the native aortic valve and radially expanded (for example, by inflating a balloon, actuating one or more actuators of the delivery apparatus, or deploying the prosthetic valve from a delivery capsule to allow the prosthetic valve to self-expand). Alternatively, a prosthetic valve can be implanted within the native aortic valve in a transapical procedure, whereby the prosthetic valve (on the distal end portion of the delivery apparatus) is introduced into the left ventricle through a surgical opening in the chest and the apex of the heart and the prosthetic valve is positioned within the native aortic valve. Alternatively, in a transaortic procedure, a prosthetic valve (on the distal end portion of the delivery apparatus) is introduced into the aorta through a surgical incision in the ascending aorta, such as through a partial J-sternotomy or right parasternal mini-thoracotomy, and then advanced through the ascending aorta toward the native aortic valve.

For implanting a prosthetic valve within the native mitral valve via a transseptal delivery approach, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, into the right atrium, across the atrial septum (through a puncture made in the atrial septum), into the left atrium, and toward the native mitral valve. Alternatively, a prosthetic valve can be implanted within the native mitral valve in a transapical procedure, whereby the prosthetic valve (on the distal end portion of the delivery apparatus) is introduced into the left ventricle through a surgical opening in the chest and the apex of the heart and the prosthetic valve is positioned within the native mitral valve.

For implanting a prosthetic valve within the native tricuspid valve, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, and into the right atrium, and the prosthetic valve is positioned within the native tricuspid valve. A similar approach can be used for implanting the prosthetic valve within the native pulmonary valve or the pulmonary artery, except that the prosthetic valve is advanced through the native tricuspid valve into the right ventricle and toward the pulmonary valve/pulmonary artery.

Another delivery approach is a transatrial approach whereby a prosthetic valve (on the distal end portion of the delivery apparatus) is inserted through an incision in the chest and an incision made through an atrial wall (of the right or left atrium) for accessing any of the native heart valves. Atrial delivery can also be made intravascularly, such as from a pulmonary vein. Still another delivery approach is a transventricular approach whereby a prosthetic valve (on the distal end portion of the delivery apparatus) is inserted through an incision in the chest and an incision made through the wall of the right ventricle (typically at or near the base of the heart) for implanting the prosthetic valve within the native tricuspid valve, the native pulmonary valve, or the pulmonary artery.

In all delivery approaches, the delivery apparatus can be advanced over a guidewire and/or an introducer sheath previously inserted into a patient's vasculature. Moreover, the disclosed delivery approaches are not intended to be limited. Any of the prosthetic valves disclosed herein can be implanted using any of various delivery procedures and delivery devices known in the art.

### Additional Examples of the Disclosed Technology

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A prosthetic valve, comprising a radially expandable frame comprising a plurality of interconnected struts, wherein the frame is radially expandable between a radially compressed state and a radially expanded state, wherein the frame comprises at least one axially oriented first post having an externally threaded portion; a valvular structure disposed within the frame and configured to regulate the flow of blood through the frame in one direction; at least one rotatable actuator operatively coupled to the frame; and wherein the actuator comprises an internally threaded bore that receives the externally threaded portion of the post, wherein the actuator is rotatable relative to the post in a first rotational direction to produce radial expansion of the frame from the radially compressed state to the radially expanded state.

Example 2. The prosthetic valve of any example herein, particularly example 1, wherein the frame comprises at least one axially oriented second post having a channel extending therethrough and wherein the actuator extends through the channel.

Example 3. The prosthetic valve of any example herein, particularly example 2, further comprising a stopper mounted on the actuator between the axially oriented first post and the axially oriented second post.

Example 4. The prosthetic valve of any example herein, particularly examples 2-3, wherein the plurality of interconnected struts form a plurality of outer cells and a plurality of inner cells, wherein each inner cell has a first apex and a second apex, and is disposed within a corresponding outer cell.

Example 5. The prosthetic valve of any example herein, particularly example 4, wherein the axially oriented first post extends axially from the first apex of an inner cell towards the second apex of the same inner cell.

Example 6. The prosthetic valve of any example herein, particularly examples 4-5, wherein the axially oriented second post extends from second apex of an inner cell to an apex of an outer cell.

Example 7. The prosthetic valve of any example herein, particularly examples 1-6, wherein the prosthetic valve comprises two or more actuators, and an equal number of corresponding axially oriented first posts having externally threaded portions, wherein each actuator comprises an internally threaded bore that receives the threaded portion of a corresponding axially oriented first post.

Example 8. The prosthetic valve of any example herein, particularly examples 1-7, wherein the radially expandable frame is formed as a unitary structure.

Example 9. The prosthetic valve of any example herein, particularly examples 1-8, wherein the radially expandable frame comprises a plastically-expandable material.

Example 10. The prosthetic valve of any example herein, particularly examples 1-9, wherein the radially expandable frame comprises a shape memory alloy.

Example 11. The prosthetic valve of any example herein, particularly example 10, wherein the shape memory alloy is nitinol.

Example 12. The prosthetic valve of any example herein, particularly examples 1-11, wherein a head portion of the actuator is configured to be releasably connected to a component of a delivery apparatus actuator.

Example 13. The prosthetic valve of any example herein, particularly examples 1-12, further comprising a sealing skirt mounted around an outer surface of the frame.

Example 14. A prosthetic valve, comprising an annular frame comprising a first end portion, a second end portion, a longitudinal axis extending from the first end portion to the second end portion, and a plurality of struts forming a plurality of cells between the first end portion and the second end portion; a valvular structure disposed within the frame and configured to regulate the flow of blood through the frame in one direction; a plurality of vertical posts oriented parallel to the longitudinal axis; and an actuation member with a first end portion and a second end portion, wherein the first end portion comprises an axially oriented, internally threaded bore; wherein the internally threaded bore of the actuation member is operatively coupled to an externally threaded portion of a first vertical post of the plurality of vertical posts; and wherein the actuation member is rotatable relative to the first vertical post in a first rotational direction to radially expand the frame from a radially compressed state to a radially expanded state and rotatable in a second rotational direction to radially compress the frame from the radially expanded state to the radially compressed state.

Example 15. The prosthetic valve of any example herein, particularly example 14, wherein the first vertical post is one of a pair of circumferentially aligned vertical posts comprising the first vertical post and a second vertical post.

Example 16. The prosthetic valve of any example herein, particularly example 15, wherein the second post comprises an axially oriented channel through which the actuation member extends.

Example 17. The prosthetic valve of any example herein, particularly examples 14-16, wherein the plurality of cells comprises a plurality of outer cells extending from the first end portion of the frame to the second end portion of the frame and a plurality of inner cells, each disposed within a corresponding outer cell.

Example 18. The prosthetic valve of any example herein, particularly example 17, wherein the first vertical post has a first end portion and a second end portion, wherein the first end portion is connected to a first vertex of an inner cell, and the first vertical post extends axially towards a second vertex of the inner cell and is operatively coupled to the actuation member at a location between the first and second vertex.

Example 19. The prosthetic valve of any example herein, particularly examples 15-18, further comprising a stopper mounted on the actuation member between the first vertical post and the second vertical post.

Example 20. The prosthetic valve of any example herein, particularly examples 14-19, wherein the prosthetic valve comprises two or more actuation members each having an axially oriented, internally threaded bore operatively coupled to an externally threaded portion of one of the axially oriented vertical posts.

Example 21. The prosthetic valve of any example herein, particularly examples 14-20, wherein the prosthetic valve comprises a shape memory alloy.

Example 22. The prosthetic valve of any example herein, particularly example 21, wherein the shape memory alloy is nitinol.

Example 23. The prosthetic valve of any example herein, particularly examples 14-22, wherein the second end of the actuation member is configured to be releasably attached to a component of a delivery apparatus.

Example 24. The prosthetic valve of any example herein, particularly examples 14-23, further comprising a sealing skirt extending around an outer surface of the frame.

Example 25. A medical assembly, comprising a prosthetic heart valve having a radially expandable frame, a valvular structure disposed within the frame and configured to regulate the flow of blood through the frame in one direction, a vertically oriented first post, and at least one actuator operatively coupled to the vertically oriented first post; a delivery device releasably attached to the actuator; wherein the frame comprises a plurality of interconnected struts and is radially expandable between a radially compressed state and a radially expanded state; wherein the vertically oriented first post comprises an externally threaded end portion; and wherein the actuator comprises an internally threaded end portion that receives the externally threaded end portion of the vertically oriented first post, wherein the actuator is rotatable relative to the first post in a first rotational direction to produce radial expansion of the frame.

Example 26. The medical of any example herein, particularly example 25, wherein and the frame comprises a vertically oriented second post comprises an axially extending channel through which the actuator extends.

Example 27. The medical assembly of any example herein, particularly examples 25-26, further comprising a stopper disposed on the actuator between the first post and the second post.

Example 28. The medical assembly of any example herein, particularly examples 25-27, wherein the prosthetic valve comprises two or more actuators and a plurality of first posts, each actuator having an axially oriented, internally threaded bore operatively connected to an externally threaded end portion of a corresponding first post.

Example 29. The medical assembly of any example herein, particularly examples 25-28, wherein the radially expandable frame comprises a plurality of outer cells arranged in an annular configuration, and a plurality of inner cells each disposed within a corresponding outer cell.

Example 30. The medical assembly of any example herein, particularly example 29, wherein the first post is disposed at least partially within an inner cell.

Example 31. The medical assembly of any example herein, particularly examples 25-30, wherein the prosthetic valve comprises a shape memory alloy.

Example 32. The medical assembly of any example herein, particularly example 31, wherein the shape memory alloy is nitinol.

Example 33. The medical assembly of any example herein, particularly examples 25-32, further comprising a sealing skirt extending around an outer surface of the frame.

Example 34. The medical assembly of any example herein, particularly examples 25-33, wherein the delivery device comprises an actuation assembly configured to be releasably connected to the actuator of the prosthetic valve, wherein the actuation assembly is configured to rotate the actuator when so coupled to the actuator.

Example 35. An expandable frame, leaflets, or prosthetic heart valve of any preceding example, wherein the expandable frame, leaflets, or prosthetic heart valve is sterilized.

Example 36. A method comprising sterilizing the prosthetic heart valve, the frame, the medical assembly, or the stent of any preceding example.

The features described herein with regard to any example can be combined with other features described in any one or more of the some examples, unless otherwise stated. For example, any one or more of the features of one frame or actuator can be combined with any one or more features of another frame or actuator.

In view of the many possible ways in which the principles of the disclosure may be applied, it should be recognized that the illustrated configurations depict examples of the disclosed technology and should not be taken as limiting the scope of the disclosure nor the claims. Rather, the scope of the claimed subject matter is defined by the following claims and their equivalents.

1. A prosthetic valve, comprising:
a radially expandable frame comprising a plurality of interconnected struts, wherein the frame is radially expandable between a radially compressed state and a radially expanded state, wherein the frame comprises at least one axially oriented first post having an externally threaded portion;
a valvular structure disposed within the frame and configured to regulate the flow of blood through the frame in one direction; and
at least one rotatable actuator operatively coupled to the frame;
wherein the actuator comprises an internally threaded bore that receives the externally threaded portion of the first post, wherein the actuator is rotatable relative to the first post in a first rotational direction to produce radial expansion of the frame from the radially compressed state to the radially expanded state.

2. The prosthetic valve of claim 1, wherein the frame comprises at least one axially oriented second post having a channel extending therethrough and wherein the actuator extends through the channel.

3. The prosthetic valve of claim 2, further comprising a stopper mounted on the actuator between the axially oriented first post and the axially oriented second post.

4. The prosthetic valve of claim 2, wherein the plurality of interconnected struts form a plurality of outer cells and a plurality of inner cells, wherein each inner cell has a first apex and a second apex, and is disposed within a corresponding outer cell.

5. The prosthetic valve of claim 4, wherein the axially oriented first post extends axially from the first apex of an inner cell towards the second apex of the same inner cell.

6. The prosthetic valve of claim 4, wherein the axially oriented second post extends from the second apex of an inner cell to an apex of an outer cell.

7. The prosthetic valve of claim 1, wherein the prosthetic valve comprises two or more actuators, and an equal number of corresponding axially oriented first posts having externally threaded portions, wherein each actuator comprises an internally threaded bore that receives the externally threaded portion of a corresponding axially oriented first post.

8. The prosthetic valve of claim 1, wherein a head portion of the actuator is configured to be releasably connected to a component of a delivery apparatus actuator.

9. The prosthetic valve of claim 1, further comprising a sealing skirt mounted around an outer surface of the frame.

10. A prosthetic valve, comprising:
an annular frame comprising a first end portion, a second end portion, a longitudinal axis extending from the first end portion to the second end portion, and a plurality of struts forming a plurality of cells between the first end portion and the second end portion;
a valvular structure disposed within the frame and configured to regulate the flow of blood through the frame in one direction;
a plurality of vertical posts oriented parallel to the longitudinal axis; and
an actuation member with a first end portion and a second end portion, wherein the first end portion comprises an axially oriented, internally threaded bore;
wherein the internally threaded bore of the actuation member is operatively coupled to an externally threaded portion of a first vertical post of the plurality of vertical posts; and
wherein the actuation member is rotatable relative to the first vertical post in a first rotational direction to radially expand the frame from a radially compressed state to a radially expanded state and rotatable in a second rotational direction to radially compress the frame from the radially expanded state to the radially compressed state.

11. The prosthetic valve of claim 10, wherein the plurality of cells comprises a plurality of outer cells extending from the first end portion of the frame to the second end portion of the frame and a plurality of inner cells, each disposed within a corresponding outer cell.

12. The prosthetic valve of claim 11, wherein the first vertical post has a first end portion and a second end portion, wherein the first end portion is connected to a first vertex of an inner cell, and the first vertical post extends axially towards a second vertex of the inner cell and is operatively coupled to the actuation member at a location between the first and second vertex.

13. The prosthetic valve of claim 10, further comprising a stopper mounted on the actuation member between the first vertical post and a second vertical post axially spaced apart from the first vertical post.

14. The prosthetic valve of claim 10, wherein the prosthetic valve comprises two or more actuation members each having an axially oriented, internally threaded bore operatively coupled to an externally threaded portion of one of the vertical posts.

15. A medical assembly, comprising:
a prosthetic heart valve having a radially expandable frame, a valvular structure disposed within the frame and configured to regulate the flow of blood through the frame in one direction, a vertically oriented first post, and at least one actuator operatively coupled to the vertically oriented first post; and
a delivery device releasably attached to the actuator;
wherein the frame comprises a plurality of interconnected struts and is radially expandable between a radially compressed state and a radially expanded state;
wherein the vertically oriented first post comprises an externally threaded end portion; and
wherein the actuator comprises an internally threaded end portion that receives the externally threaded end portion of the vertically oriented first post, wherein the actuator is rotatable relative to the vertically oriented first post in a first rotational direction to produce radial expansion of the frame.

16. The medical assembly of claim 15, wherein and the frame comprises a vertically oriented second post comprises an axially extending channel through which the actuator extends.

17. The medical assembly of claim 16, further comprising a stopper disposed on the actuator between the vertically oriented first post and the vertically oriented second post.

18. The medical assembly of claim 15, wherein the prosthetic heart valve comprises two or more actuators and a plurality of first posts, each actuator having an axially oriented, internally threaded bore operatively connected to an externally threaded end portion of a corresponding first post.

19. The medical assembly of claim 15, wherein the radially expandable frame comprises a plurality of outer cells arranged in an annular configuration, and a plurality of inner cells each disposed within a corresponding outer cell.

20. The medical assembly of claim 15, wherein the delivery device comprises an actuation assembly configured to be releasably connected to the actuator of the prosthetic heart valve, wherein the actuation assembly is configured to rotate the actuator when so coupled to the actuator.
